(19) United States
(12) Patent Application Publication
(10) Pub. No.: US 2011/0280912 A1
(43) Pub. Date: Nov. 17, 2011

(54) DEGRADABLE POLYMER NANOSTRUCTURE MATERIALS

(75) Inventors: Robert S. Langer, Newton, MA (US); Ana Jaklenec, Cambridge, MA (US); Daniel Alan Heller, Boston, MA (US); Daniel Griffith Anderson, Sudbury, MA (US); Michael S. Strano, Lexington, MA (US)

(73) Assignee: Massachusetts Institute of Technology, Cambridge, MA (US)

(21) Appl. No.: 12/969,558

(22) Filed: Dec. 15, 2010

Related U.S. Application Data
(60) Provisional application No. 61/286,764, filed on Dec. 15, 2009.

(51) Int. Cl. A61K 9/00; C08L 47/00; A61K 35/12; C08L 67/02; C08L 67/00; B82Y 5/00

(52) U.S. Cl. 424/400; 524/602; 524/607; 524/606; 524/548; 524/600; 424/93.7; 977/762; 977/906

(57) ABSTRACT

This invention relates generally to composites comprising a plurality of nanostructures, and methods of making the same. In some embodiments, the composites further comprise a polymer. In some embodiments, the composites may have desirable properties such as, for example, biodegradability, biocompatibility, and/or high tensile strength. In one embodiment, the plurality of nanostructures comprises carbon nanotubes, and the polymer comprises a poly(beta-amino ester). Various methods are provided for preparing the composites. For example, the polymer and the plurality of nanostructures may, in some embodiments, be combined in a layer-by-layer process to form the composite. High throughput methods for preparing composites having different compositions also are provided for screening composites for desirable properties.

### RELATED APPLICATIONS

This application claims priority under 35 U.S.C. §119(e) to U.S. Provisional Patent Application No. 61/286,764, filed Dec. 15, 2009, and entitled “Degradable Polymer Nanostructures,” which is incorporated herein by reference in its entirety for all purposes.

### GOVERNMENT FUNDING

Research leading to various aspects of the present invention were sponsored, at least in part, by the National Institutes of Health, grant number 5F32AR055438. The U.S. Government has certain rights in the invention.

### FIELD OF INVENTION

This invention relates generally to composites and other compositions comprising a plurality of nanostructures, and methods of making the same. In some embodiments, the compositions further comprise a polymer.

### BACKGROUND

Biodegradable materials have been used to fabricate composites for medical applications. However, a need exists for biodegradable composites and other compositions with enhanced mechanical and/or other characteristics.

### SUMMARY OF INVENTION

This invention relates generally to composites and other compositions comprising a plurality of nanostructures, and methods of making the same. In some embodiments, the compositions further comprise a polymer. The subject matter of the present invention involves, in some cases, interrelated products, alternative solutions to a particular problem, and/or a plurality of different uses of one or more compositions and/or articles.

In one aspect, a composite is provided. In some embodiments, the composite comprises a polymer comprising a poly(beta-amino ester); and a plurality of nanostructures contained within a volume of the polymer, wherein the yield strength and/or effective Young's modulus of the composite is substantially greater than that which would be observed in the absence of the nanostructures, but under otherwise substantially identical conditions.

In another aspect, an article is provided. The article can comprise a fluid; a polymer comprising a poly(beta-amino ester) contained within the fluid; and a nanostructure contained within the fluid and interacting with the polymer.

In one aspect, a method is provided. In some embodiments, the method comprises providing a fluid; distributing, within the fluid, a polymer comprising a poly(beta-amino ester); and distributing, within the fluid, a plurality of nanostructures.

In some embodiments, the method comprises providing a polymer comprising a biodegradable poly(beta-amino ester); and distributing, within a volume of the polymer, a plurality of nanostructures, wherein the polymer and nanostructures are together selected such that the composite biodegrades over a predetermined period of time.

In some embodiments, the method comprises providing a polymer comprising a poly(beta-amino ester); and distributing, within the polymer, a plurality of nanostructures, wherein the polymer and nanostructures are together selected such that the composite has a predetermined yield strength and/or effective Young's modulus prior to first use.

In one aspect, a kit is provided. The kit comprises, in some embodiments, a composite, comprising a polymer comprising a poly(beta-amino ester); and a plurality of nanostructures within a volume of the polymer.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows diacrylate monomers used for library synthesis, according to various embodiments;

FIG. 1B shows amine monomers for library synthesis, according to various embodiments;

FIG. 2A shows a UV-vis spectrum of polymer-wrapped SWNT, according to various embodiments;

FIG. 2B shows a fluorescence spectrum of polymer-wrapped SWNT, according to various embodiments;

FIG. 3A shows the structure of polymers that can be used to wrap a SWNT in the presence of water, according to various embodiments;

FIG. 3B shows the structure of polymers that can be used to wrap a SWNT in the presence of acetonitrile, according to various embodiments; and

FIG. 4 includes the structure of a polymer used to wrap a SWNT, according to one set of embodiments.

### DETAILED DESCRIPTION

This invention relates generally to composites comprising a plurality of nanostructures, and methods of making the same. In some embodiments, the composites further comprise a polymer. In some embodiments, the composites may have desirable properties such as, for example, biodegradability, biocompatibility, and/or high tensile strength. In one embodiment, the plurality of nanostructures comprises carbon nanotubes, and the polymer comprises a poly(beta-amino ester). Various methods are provided for preparing the composites. For example, the polymer and the plurality of nanostructures may, in some embodiments, be combined in a layer-by-layer process to form the composite. High throughput methods for preparing composites having different compositions also are provided for screening composites for desirable properties.

In some cases, a composite of the invention may exhibit a higher mechanical strength (e.g., yield strength), effective Young's modulus, and/or toughness (as measured using standard methods known to those of ordinary skill in the art such as those described in Example 3) when compared to an essentially identical material lacking nanostructures, under essentially identical conditions. In some cases, a composite may exhibit a higher thermal and/or electrical conductivity when compared to an essentially identical material lacking nanostructures, under essentially identical conditions. In some cases, the thermal, electrical conductivity, and/or other properties (e.g., electromagnetic properties, specific heat, etc.) may be anisotropic.

In some embodiments, a medical device comprising a polymer and a plurality of nanostructures is provided. The polymer and the plurality of nanostructures may form a composite. Non-limiting examples of medical devices include stents, tissue scaffolds, bandages, hernia repair devices, drug release depots, coatings for medical devices, etc. Medical devices formed from the polymers and plurality of nanostructures herein may be particularly advantageous in applications that require high yield strength, stiffness, and/or toughness, although medical devices formed from the polymers and plurality of nanostructures herein are not limited to these applications. In some embodiments, a medical device may be degradable. As discussed in more detail below, a composite may be configured for controlled release of an active agent, for example, a pharmaceutical agent.

In one aspect, composites comprising nanostructures are provided. In some embodiments, the nanostructures may be essentially uniformly dispersed within a composite, which may facilitate formation of composites having improved mechanical, thermal, electrical, or other properties. In other embodiments, a composite may have a first region that includes nanostructures and a second region that does not include nanostructures. For example, a composite may comprise a plurality of layers, where at least one layer comprises nanostructures, and at least one layer does not comprise nanostructures. In another example, a composite may have a first region that includes nanostructures at a first concentration and a second region that includes nanostructures at a second concentration, where the first concentration is greater than the second concentration.

As used herein, the term “nanostructure” refers to articles having at least one cross-sectional dimension of less than about 1 micron. In some embodiments, nanostructures can have at least one cross-sectional dimension of less than about 500 nm, less than about 250 nm, less than about 100 nm, less than about 75 nm, less than about 50 nm, less than about 25 nm, less than about 10 nm, or, in some cases, less than about 1 nm. Examples of nanostructures include nanotubes [e.g., carbon nanotubes, including single-walled carbon nanotubes (SWNT)], nanowires (e.g., carbon nanowires), nanofibers, graphene, and quantum dots, among others. In some embodiments, the nanostructures include a fused network of atomic rings, the atomic rings comprising a plurality of double bonds.

In some embodiments, a plurality of the nanostructures may be contained within a volume of polymer (i.e., the nanostructures and polymer may form a composite). A composite may comprise a mixture of cationic and anionic polymers. In some embodiments, complexation may occur between the cationic polymer and the anionic polymer resulting in precipitation of the polymer complex. As discussed in more detail below, the polymer may be, in some embodiments, a poly(beta-amino ester).

Not wishing to be bound by any particular theory, the use of polymers comprising a poly(beta-amino ester) might be advantageous for one or more reasons. In some embodiments, the poly(beta-amino ester) can be charged (e.g., protonated), for example, at amine sites, which might enhance the level to which it is able to interact with the nanostructures. The poly(beta-amino ester) can include, in some cases, at least one relatively hydrophilic portion and at least one relatively hydrophobic portion, which might give the poly(beta-amino ester) surfactant properties. As used herein, a “polymer having surfactant properties” means a polymer that can reduce the interfacial tension between a fluid and a nanostructure.

In some cases, the poly(beta-amino ester) can comprise one or more aromatic rings, which might enhance its interaction with one or more nanostructures (e.g., via pi-pi stacking, which is a phenomenon understood to those of ordinary skill in the art). Of course, the invention is not so limited and, in some cases, the polymer might comprise a relatively low amount of aromatic rings and still be effective in the embodiments described herein. In some embodiments, at least one of the repeating units within the polymer is non-aromatic (i.e., free of aromatic rings). In some cases, the polymer contains a relatively small amount of aromatic rings (e.g., less than about 5 wt %, less than about 1 wt %, less than about 0.5 wt %, less than about 0.1 wt %, less than about 0.05 wt %, or less than about 0.01 wt % aromatic rings, wherein “wt %” signifies a percentage by weight). One of ordinary skill in the art would be capable of calculating the weight percentage of aromatic rings within a polymer by dividing the sum of the molecular weights of the aromatic rings within the polymer by the overall molecular weight of the polymer. In some embodiments, the entire polymer can be non-aromatic (i.e., the polymer is free of aromatic rings).

In some embodiments, a nanostructure and a polymer may interact with each other. The interaction may occur, in some cases, via van der Waals forces (e.g., physisorption) and/or hydrogen bonding. In some embodiments, the interacting nanostructure and the polymer are not covalently bonded to each other. Accordingly, in some cases, the interaction between the polymer and the nanostructure is reversible without breaking any covalent bonds. In some embodiments, the interaction between the polymer and the nanostructure is reversible via dialysis.

In some embodiments, the interaction between a polymer and a nanostructure is such that, under set conditions, at least a portion of the polymer and at least a portion of the nanostructure move together as a unit. For example, at least a portion of the polymer can be immobilized with respect to at least a portion of the nanostructure.

The polymer may assume any suitable shape or conformation when interacting with the nanostructure. In some embodiments, the polymer may at least partially surround (i.e., wrap) the nanostructure. A first entity is said to “at least partially surround” a second entity if a closed loop can be drawn around the second entity through only the first entity. In some cases, the polymer can be oriented such that it winds around the nanostructure in a helical configuration. In some embodiments, a polymer and a nanostructure may interact at one or more locally confined regions of the nanostructure and/or polymer (e.g., at one or more points on the nanostructure and/or polymer). In some cases, the polymer may be positioned proximate to the nanostructure such that it completely surrounds the nanostructure with the exception of relatively small volumes. A first entity is said to “completely surround” a second entity if closed loops going through only the first entity can be drawn around the second entity regardless of direction. Without wishing to be bound by any theory, it is believed that polymers and nanostructures that participate in strong interactions can be used to create composites with enhanced physical properties (i.e., mechanical properties, electrical and/or thermal conductive properties, degradation properties, etc.).

In some embodiments, a cationic polymer interacts with the nanostructure. For example, the cationic polymer may wrap the nanostructure. In other embodiments, an anionic polymer may wrap the nanostructure. As discussed in more detail below, a composite structure may be formed, in some embodiments, by precipitating wrapped nanostructures from a fluid.

In some embodiments, the nanostructure may be substantially free of covalent bonds between one or more of the atoms forming the nanostructure and one of more of the atoms of other entities (e.g., other nanostructures, a polymer, the surface of a container, etc.). The absence of covalent bonding between the nanostructure and another entity may, for example, preserve one or more properties of the nanostructure. As a specific example, a composite comprising single-walled carbon nanotubes may have reduced mechanical strength if at least some of the single-walled carbon nanotubes are covalently bonded to another entity. As another specific example, a composite comprising single-walled carbon nanotubes may have reduced near-infrared fluorescence if at least some of the single-walled carbon nanotubes are covalently bonded to another entity. The embodiments described herein are not limited to non-covalent interactions between the polymer and the nanostructure, and, in some embodiments, the polymer and the nanostructure can be covalently bonded.

In some embodiments, the ratio of polymer to nanostructure (by weight) may be between about 200:1 and about 5:1, between about 100:1 and about 10:1, between about 500:1 and about 10:1, between about 1000:1 and about 10:1, between about 1000:1 and about 1:1, between about 200:1 and about 1:1, between about 100:1 and about 1:1, or between about 50:1 and about 1:1.

In some embodiments, the polymer may be crosslinked, for example to produce a composite. For example, polymers having a primary and/or secondary amine group can be crosslinked using a crosslinking agent (e.g, glutaraldehyde). In some embodiments, polymers (e.g., polymers with carbon-carbon termination) can be crosslinked using ultraviolet radiation. In some embodiments, two polymer strands, each interacting with a different nanostructure, can be crosslinked thereby linking the two nanostructures. In some cases, crosslinking a polymer in a composite can improve the mechanical strength of the composite. In some embodiments, the length of nanostructures may be chosen such that the nanostructures are capable of interacting (e.g., entangling) with one another. In this way, additional improvements to the mechanical properties of a composite may be achieved. In some cases, nanostructures may be substantially aligned in a composite. In some cases, nanostructures may be partially aligned or substantially randomly aligned in a composite. In some cases, the electrical conductivity, thermal conductivity, and/or other properties of a composite structure may also be enhanced or made anisotropic by the structures and methods of the invention.

In some embodiments, a composite may have a thin film structure. For example, as discussed in more detail below, a film may be prepared using a layer-by-layer deposition method resulting in a structure having a plurality of layers. In some embodiments, all of the layers comprise nanostructures. In other embodiments, only some of the layers comprise nanostructures. Random layers may comprise nanostructures. Alternatively, a film may be prepared having a repeating pattern of layers without nanostructures and layers comprising nanostructures. For example, in some embodiments, every layer or every other layer of the composite structure contains nanostructures. In other embodiments, every third, every fourth, every fifth layer, every sixth layer, every seventh layer, or every eighth layer comprises nanostructures, for example, by having the nanostructures interact with both cationic and anionic polymers. In some embodiments, composites may have a fiber structure.

Methods of the invention may be useful for producing composites having one or more enhanced properties, such as mechanical strength. In some embodiments, the integrity of the reinforcement may depend on the diameter and/or length of the nanostructures (e.g., nanotubes). In some embodiments, the nanostructures used in the inventive articles and methods can be selected such that they have appropriate dimensions to enhance the properties of one or more materials. In some cases, the nanostructures may have a diameter of 100 nm or less, or, in some cases, 10 nm or less. In some embodiments, the mechanical properties (e.g., effective Young's modulus, toughness, yield strength, etc.) observed for composites comprising nanostructures may be greater than those that would be observed in the absence of the nanostructures, but under otherwise substantially identical conditions, by at least 50%, 100%, 250%, 500%, 1000%, 2000%, or 3,000%. Even greater improvements in mechanical properties may be observed.

In some embodiments, a composite comprising nanostructures may have an effective Young's modulus of at least 200 MPa, 1 GPa, 2 GPa, 5 GPa, 10 GPa, 20 GPa, 30 GPa, or 50 GPa. In some embodiments, a composite comprising nanostructures may have a yield strength of at least 50 MPa, at least 100 MPa, at least 200 MPa, at least 500 MPa, at least 1 GPa, at least 2 GPa, at least 5 GPa, at least 10 GPa, or even greater. In some embodiments, the polymer and plurality of nanostructures may be selected such that the composite has a predetermined effective Young's modulus and/or yield strength prior to first use.

In some embodiments, a composite comprising nanostructures may have improved electrical conductivity in comparison to a composite prepared without nanostructures in an essentially identical manner. In some cases, the composite comprising nanostructures may display semiconductive properties.

It has been unexpectedly discovered that the composites described herein can exhibit one or more desirable properties (e.g., mechanical properties, electrical properties, and/or thermal properties) when the loading of nanostructures (e.g., carbon-based nanostructures such as carbon nanotubes) is relatively low. For example, in some embodiments, the composite can comprise, by weight, less than about 5%, less than about 1%, less than about 0.5%, less than about 0.1%, less than about 0.05%, less than about 0.01%, between about 0.001% and about 5%, between about 0.001% and about 1%, between about 0.001% and about 0.5%, between about 0.001% and about 0.1%, between about 0.001% and about 0.05%, or between about 0.001% and about 0.01%.

Composites may be biodegradable, and the degradation time can be controlled to provide a composite that degrades within a desired timeframe. In some embodiments, within a pH range of 7-8, the amount of time needed for greater than 90% of the hydrolyzable bonds within a biodegradable composite to degrade is greater than 5 days, greater than 10 days, greater than 30 days, greater than 60 days, greater than 90 days, greater than 180 days, or greater than 360 days. Those of ordinary skill in the art will recognize that hydrolyzable composites in solutions having a pH outside this range (i.e., more acidic than pH 7 or more basic than pH 8) will degrade faster than hydrolyzable composites exposed to essentially identical conditions but within a pH range of 7-8. The degradation time can be increased, in some embodiments, generally by selecting a monomer for incorporation into the polymer that increases the hydrophobicity of the polymer and/or decreases the electrophilicity of at least some of the hydrolyzable bonds in the polymer. Generally, monomers that decrease the hydrophobicity of the polymer and/or increase the electrophilicity of at least some of the hydrolyzable bonds in the polymer can be used to decrease the degradation time of the polymer. Those of ordinary skill in the art will readily be able to select monomers that can increase or decrease the degradation time by routine experimentation.

In another aspect, a method of making a composite is provided. The method of making the composite may comprise, in some cases, exposing a nanostructure to a polymer capable of interacting with the nanostructure (e.g., via any of the mechanisms described above). In some embodiments, the nanostructure, the polymer, or both may be provided within a fluid (e.g., a liquid). For example, exposing a nanostructure to the polymer can comprise adding the polymer to a fluid containing a nanostructure. Exposing a nanostructure to a polymer can also comprise adding a nanostructure to a fluid containing a polymer, in some cases. One of ordinary skill in the art will be able to identify other suitable methods for exposing a nanostructure to a polymer. In some embodiments, the polymer and the nanostructure interact with each other when they are in the fluid, for example, via any of the mechanisms described herein and/or to produce any of the nanostructure/polymer arrangements described herein. In some embodiments, a method of forming the composite comprises a complexation between at least two polymers. In one embodiment, the first polymer interacts with a nanostructure and a second polymer complexes with the first polymer to form a precipitate.

As used herein, the term “fluid” generally refers to a substance that tends to flow and to conform to the outline of its container. Typically, fluids are materials that are unable to withstand a static shear stress, and when a shear stress is applied, the fluid experiences a continuing and permanent distortion. The fluid may have any suitable viscosity that permits at least some flow of the fluid. Non-limiting examples of fluids include liquids and gases, but may also include free-flowing solid particles (e.g., cells, vesicles, etc.), viscoelastic fluids, and the like.

A variety of fluids can be used in association with the inventive articles, systems, and methods described herein. In some embodiments, the fluid may comprise water. In some cases, an organic fluid can be used such as, for example, chloroform, acetonitrile, butanol, DMF, N-methylpyrrolidone (NMP), any other suitable fluid in which nanostructures (e.g., carbon nanotubes) and/or a polymer (e.g., a poly(beta-amino ester) can be suspended, and/or mixtures of these. In some embodiments, a fluid may be selected that is capable of forming a stable suspension of nanostructures (e.g., single-walled carbon nanotubes).

In some embodiments, the polymer can be solidified to form a composite structure. Solidification can be achieved, for example, via further polymerization of the polymer, cross-linking of the polymer, removal of the fluid in which the polymer is suspended (e.g., via drying, filtering, and the like), or by any other suitable method.

In some embodiments, formation of a composite may further comprise complexation between two polymers. For example, a first polymer may interact with a nanostructure (e.g., by forming a wrapped structure), and a second polymer may interact with the first polymer to form a precipitate. In some embodiments, the first polymer is a cationic polymer, and the second polymer is an anionic polymer. In other embodiments, the first polymer is an anionic polymer, and the second polymer is a cationic polymer.

In some embodiments, a film composite may be formed by depositing thin films of polymer or a mixture of polymer and nanostructures. The thin films may be formed by any method known in the art, for example, dip coating or spin coating a fluid having a polymer or a mixture of polymer and nanostructures distributed therein onto a substrate. The substrate may be any suitable substrate. Non-limiting examples of substrates include glass, polyethylene, Teflon, and silicon. In one embodiment, a film may be formed by coating a substrate with a cationic polymer [e.g., a poly(beta-amino ester)] to form a first layer, coating the first layer with an anionic polymer (e.g., polyacrylic acid, alginic acid, chondroitin sulfate, dextran sulfate, or heparin) to form a second layer, coating the second layer with a mixture of a cationic polymer and a plurality of nanostructures to form a third layer, and coating the third layer with an anionic layer to form a fourth layer. This process may be repeated until a film of the desired thickness is achieved. In some embodiments, a rinse step may be included between one or more layer formation steps. One of ordinary skill in the art would readily recognize that the order of the layers may be varied.

The polymer and/or mixture of polymer and nanostructures may be solidified on the substrate and/or layer such that the nanostructures are contained within the volume of the polymer. Solidification may occur in a variety of ways. For example, the polymer may precipitate from the fluid, the fluid may be evaporated, etc.

The completed film may be removed from the substrate by any suitable method. For example, in some embodiments, a film may be removed from a substrate by peeling the film off the substrate. In one example, a film may be removed by dissolving the substrate. For example, a film may be removed from a glass substrate by dissolving the glass in hydrofluoric acid.

In some embodiments, a fiber may be formed. A fiber may be formed by any suitable method, for example, using a single-phase system or a two-phase system. In a single-phase system, a fluid may comprise at least two polymers and a plurality of nanostructures. Fibers in such a system may be formed by agitation, for example, by stirring the system with a rod. In a two-phase system, a first polymer is provided in a first fluid and a second polymer is provided in a second fluid, where the first fluid and the second fluid are essentially immiscible. A plurality of nanostructures may be provided in the first fluid, the second fluid, or both the first fluid and the second fluid. At the interface of the two fluids, an essentially instantaneous complexation may occur resulting in the formation of a fiber. The fiber may be pulled from the system in a controlled fashion such that a continuous fiber is formed with nascent fiber being continuously formed at the interface.

A variety of polymers can be used in the embodiments described herein. In some embodiments, the polymer may be biodegradable and/or biocompatible. For example, the polymer may comprise a polyester, a polyanhydride, or a polycarbonate. For example, the polymer may be poly(glycolide-co-lactide) (PLGA), polyglycolic acid, polylactide, or polycaprolactone. One of ordinary skill in the art would readily be able to identify biodegradable polymers in the art. The polymer may comprise blends, mixtures, and/or copolymers. In some embodiments, the polymer is anionic. In some embodiments, the polymer is cationic. In one embodiment, the polymer comprises a poly(beta-amino ester).

Non-limiting examples of polymers that may be used with the present invention are disclosed in U.S. Pat. No. 6,998,115, entitled “Biodegradable Poly(β-amino esters) and Uses Thereof,” issued Feb. 14, 2006; U.S. patent application Ser. No. 11/758,078, filed Jun. 5, 2007, entitled “Crosslinked, Degradable Polymers and Uses Thereof,” published as U.S. Patent Application Publication No. 2008/0145338 on Jun. 19, 2008; U.S. Provisional Patent Application No. 61/286,764, filed Dec. 15, 2009, and entitled “Degradable Polymer Nanostructures;” U.S. Pat. No. 7,427,394, entitled “Biodegradable Poly(Beta-Amino Esters) and Uses Thereof,” issued Sep. 23, 2008; U.S. patent application Ser. No. 11/780,754, filed Jul. 21, 2006, entitled “End-Modified Poly(beta-amino esters) and Uses Thereof,” published as U.S. Patent Application Publication No. 2008/0242626 on Oct. 2, 2008; U.S. patent application Ser. No. 11/099,886, filed Apr. 6, 2005, entitled “Biodegradable Poly(Beta-Amino Esters) and Uses Thereof,” published as U.S. Patent Application Publication No. 2005/0265961 on Dec. 1, 2005; U.S. patent application Ser. No. 12/568,481, Sep. 28, 2009, entitled “Biodegradable Poly(Beta-Amino Esters) and Uses Thereof”; and U.S. patent application Ser. No. 12/833,749, filed Jul. 9, 2010, entitled “Biodegradable Poly(Beta-Amino Esters) and Uses Thereof, each of which is incorporated herein by reference in its entirety for all purposes.

In some embodiments, the polymer contains a tertiary amine in the polymer backbone. The polymer molecular weight may range, in some embodiments, from 5,000 g/mol to 100,000 g/mol or from 4,000 g/mol to 50,000 g/mol. In one embodiment, the polymer may be essentially non-cytotoxic. In one embodiment, the polymer may have a pKa between 5.5 to 7.5 or between 6.0 and 7.0. In another embodiment, the polymer may be designed to have a desired pKa between 3.0 and 9.0 or between 5.0 and 8.0.

A poly(beta-amino ester) can generally be defined by the formula (I):

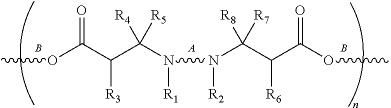

The linkers A and B are each a chain of atoms covalently linking the amino groups and ester groups, respectively. These linkers may contain carbon atoms or heteroatoms (e.g., nitrogen, oxygen, sulfur, etc.). Typically, these linkers are 1 to 30 atoms long or 1 to 15 atoms long. The linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted. The groups R₁, R₂, R₃, R₄, R₅, R₆, R₇, and R₈ may be any chemical groups including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, alkylthioether, thiol, and ureido groups. “n” may be an integer ranging from 2 to 10,000, from 5 to 10,000, or from 10 to 500. In some embodiments, “n” may be an integer greater than 2, greater than 5, greater than 10, greater than 50, or greater than 100. It should be understood that “n” may be an integer in a range outside of these ranges as well.

In one embodiment, the poly(beta-amino ester) may be generally represented by the formula II:

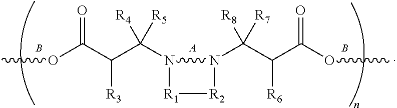

In this embodiment, R₁ and R₂ are directly linked together as shown in formula II. As described above in the preceding paragraph, any chemical group that satisfies the valency of each atom may be substituted for any hydrogen atom.

In another embodiment, the groups R₁ and/or R₂ may be covalently bonded to linker A to form one or two cyclic structures. In some embodiments, the poly(beta-amino ester) may be represented by the formula V in which both R₁ and R₂ are bonded to linker A to form two cyclic structures:

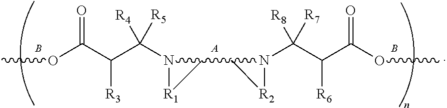

The cyclic structures may be 3-, 4-, 5-, 6-, 7-, or 8-membered rings or larger. The rings may contain heteroatoms and be unsaturated. As described above, any chemical group that satisfies the valency of each atom in the molecule may be substituted for any hydrogen atom.

In another embodiment, the poly(beta-amino ester) can generally be defined by the formula (IX):

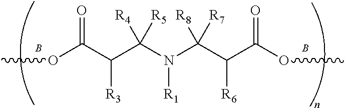

The linker B is a chain of atoms covalently linking the ester groups. The linker may contain carbon atoms or heteroatoms (e.g., nitrogen, oxygen, sulfur, etc.). In some embodiments, the linker may be 1 to 30 atoms long or 1-15 atoms long. The linker may be substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted. Each of R₁, R₃, R₄, R₅, R₆, R₇, and R₈ may be independently any chemical group including, but not limited to, hydrogen atom, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, alkylthioether, thiol, and ureido groups. “n” may be an integer ranging from 2 to 10,000, from 5 to 10,000, or from 10 to 500. In some embodiments, “n” may be an integer greater than 2, greater than 5, greater than 10, greater than 50, or greater than 100. It should be understood that “n” may be an integer in a range outside of these ranges as well. In some embodiments, R₃, R₄, R₅, R₆, R₇, and R₈ are all hydrogen.

In another embodiment, a bis(acrylate ester) unit in the poly(beta-amino ester) is chosen from the following group of bis(acrylate ester) units:

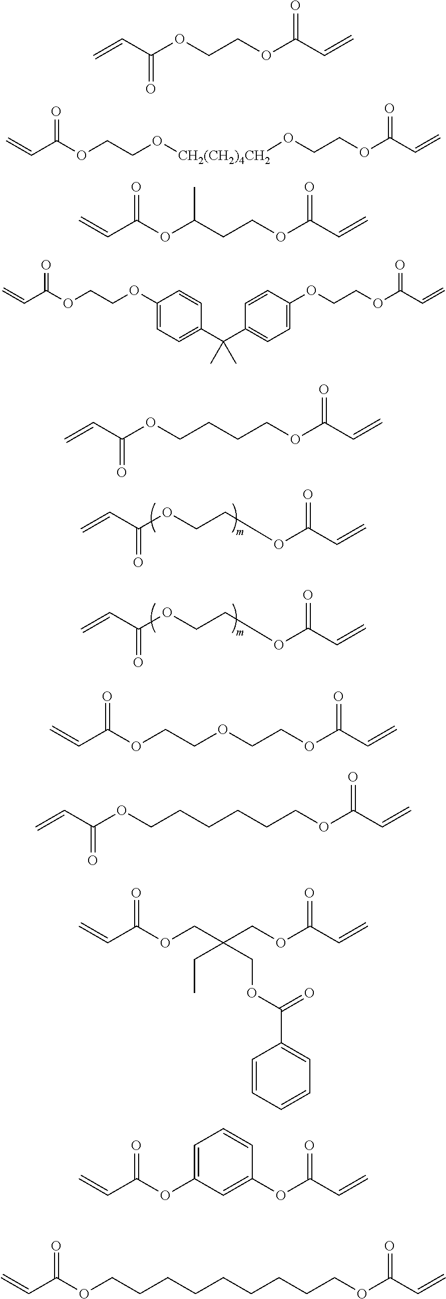

“m” may be an integer ranging from 2 to 10,000, from 5 to 10,000, or from 10 to 500. In some embodiments, “m” may be an integer greater than 2, greater than 5, greater than 10, greater than 50, or greater than 100. It should be understood that “m” may be an integer in a range outside of these ranges as well.

In another embodiment, an amine in the poly(beta-amino ester) is chosen from the following group of amines:

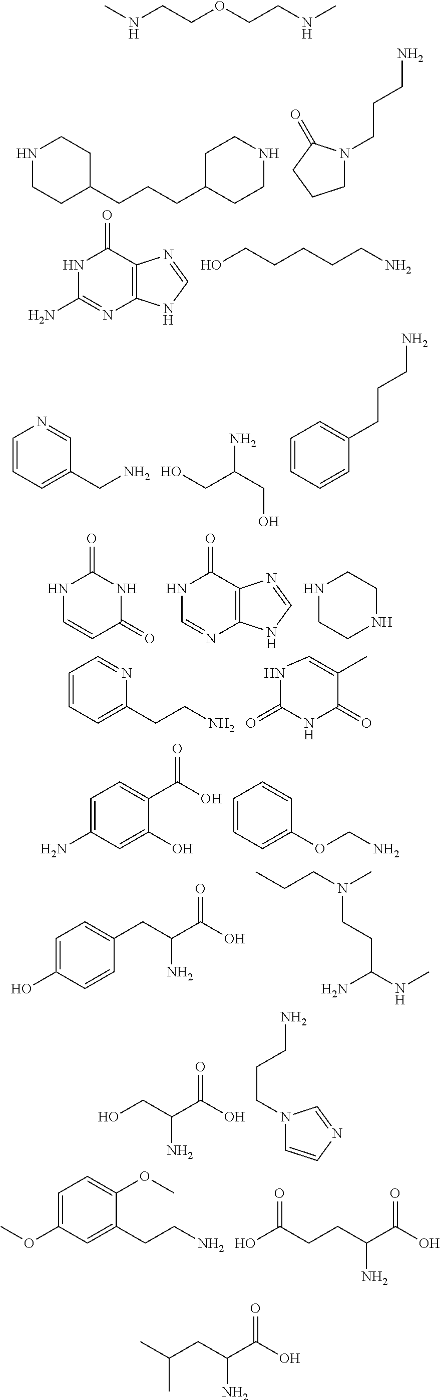

Non-limiting examples of poly(beta-amino esters) include:

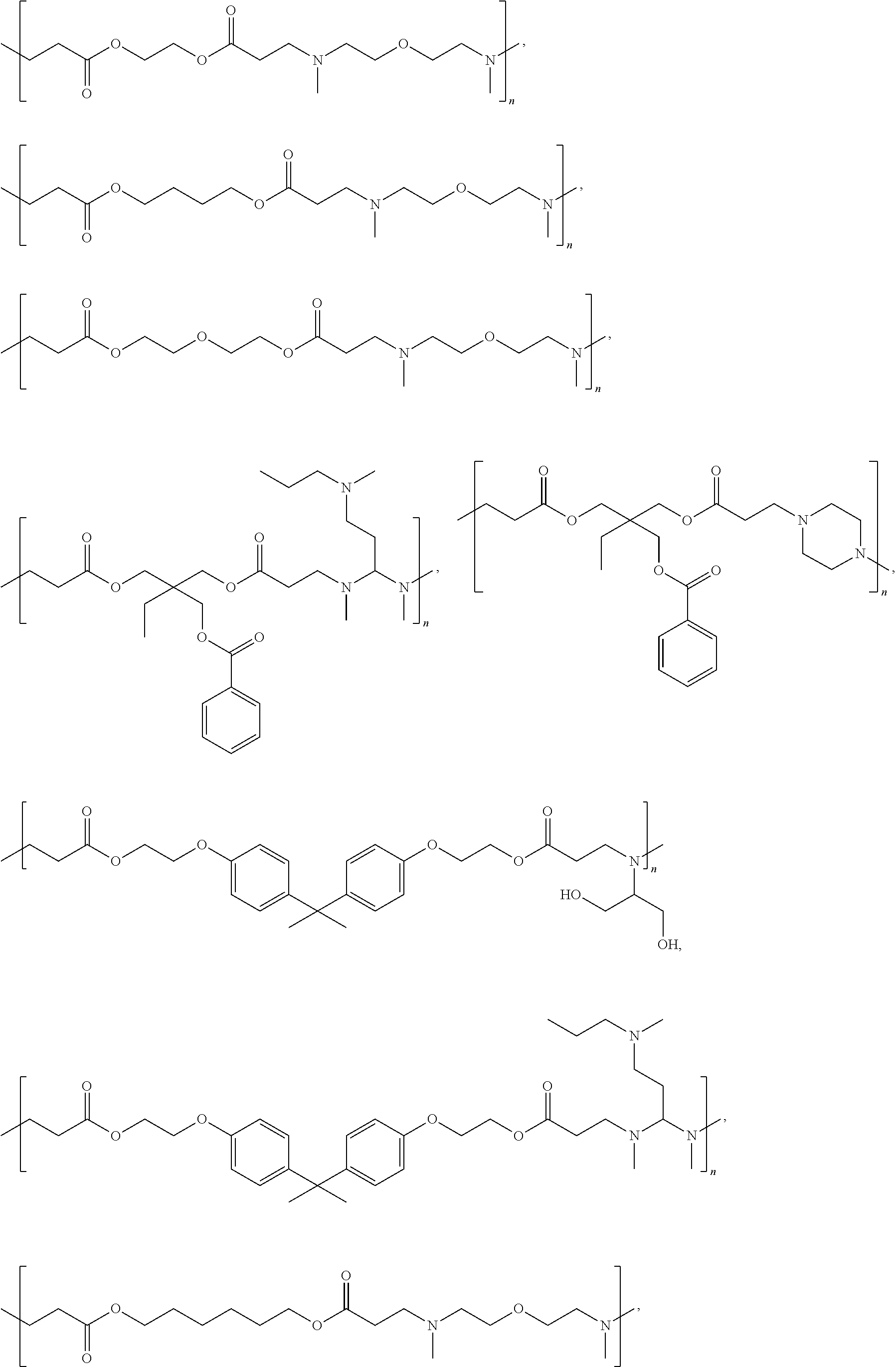

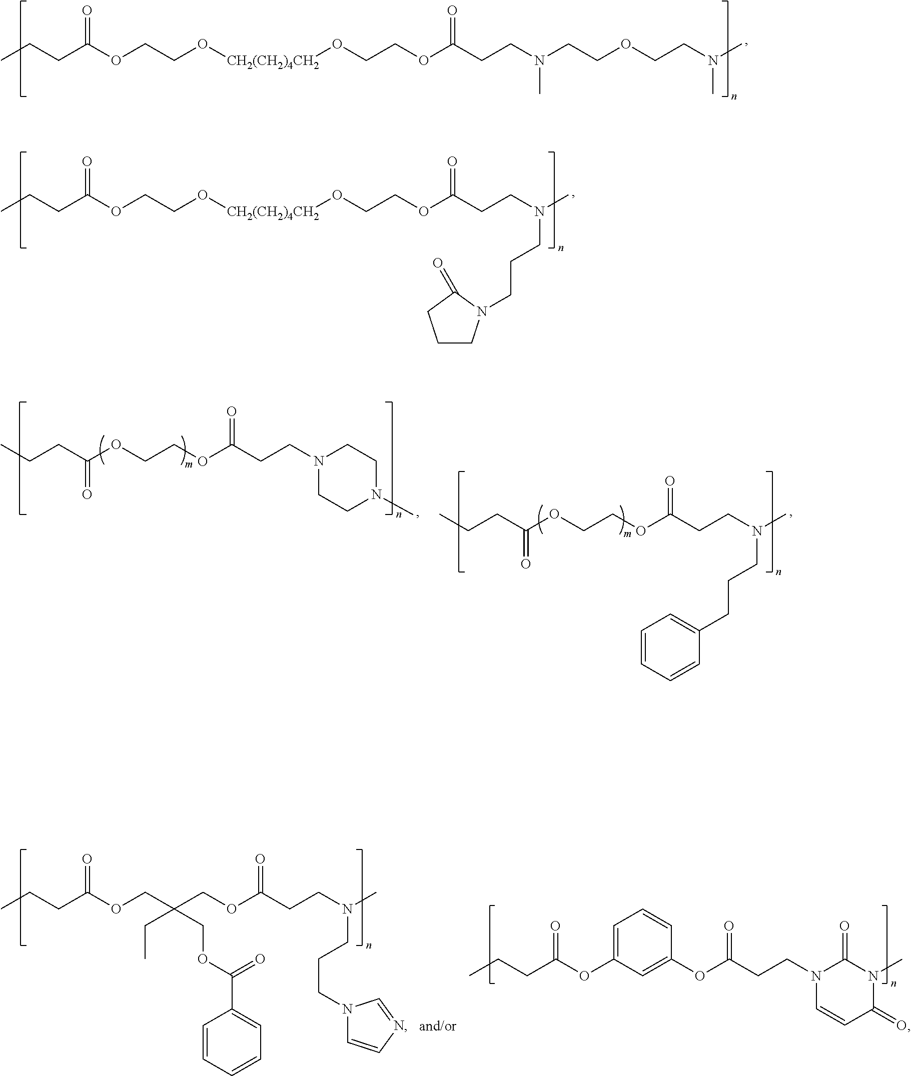

where “m” may be an integer ranging from 2 to 10,000, from 5 to 10,000, or from 10 to 500, or may be an integer greater than 2, greater than 5, greater than 10, greater than 50, or greater than 100, and where “n” may be an integer ranging from 2 to 10,000, from 5 to 10,000, or from 10 to 500, or may be an integer greater than 2, greater than 5, greater than 10, greater than 50, or greater than 100. It should be understood that “n” may be an integer in a range outside of these ranges as well. It should also be understood that “m” may be an integer in a range outside of these ranges as well.

In some embodiments, the polymer comprises a non-degradable polymer (e.g., polyvinyl, poly(acrylic acid), polymethacrylate, poly(ethylene oxide), poly(vinyl pyrrolidinone), poly(allyl amine), poly(2-vinylpyridine), poly(maleic acid), and the like). In some embodiments, the polymer may comprise a polysaccharide. For example, the polymer may comprise dextran, amylose, chitin, heparin, hyaluronic acid, or cellulose. In some embodiments, the polymer may comprise a protein. Examples of suitable proteins include, but are not limited to glucose oxidase, bovine serum albumin, and alcohol dehydrogenase. In some embodiments, the polymer may comprise a polynucleotide. For example, the polymer may comprise a series of repeated base pairs (e.g., repeated adenine-thymine (AT) base pairs, repeated guanine-thymine (GT) base pairs, etc.) In some embodiments, the polymer may comprise at least about 5, at least about 15, at least about 25, at least about 50, or at least about 100, between 5 and 30, or between 10 and 20, or about 15 repeated base pairs (e.g., AT, GT, and the like) in succession. In one embodiment, the polymer of the present invention is a co-polymer wherein one of the repeating units is a poly(beta-amino ester).

A polymer may be prepared by any method known in the art. In some embodiments, the polymers are prepared from commercially available starting materials. In another embodiment, the polymers are prepared from easily and/or inexpensively prepared starting materials.

In one embodiment, a poly(beta-amino ester) may be prepared by conjugate addition of bis(secondary amines) to bis(acrylate ester). In another embodiment, a poly(beta-amino ester) may be prepared by conjugate addition of a primary amine to a bis(acrylate ester).

In some embodiments, each of the monomers may be dissolved in an organic solvent (e.g., THF, CH₂Cl₂, MeOH, EtOH, CHCl₃, hexanes, toluene, benzene, CCl₄, diethoxymethane, diethyl ether, etc.). In some embodiments, the resulting solutions may be combined, and the reaction mixture may be heated to yield the desired polymer. In one embodiment, the reaction mixture is heated to approximately 50° C. In another embodiment, the reaction mixture may be heated to approximately 75° C. In still another embodiment, the reaction mixture may be maintained at 20° C. The reaction mixture may also be cooled to approximately 0° C. The polymerization reaction may also be catalyzed. As would be appreciated by one of ordinary skill in the art, the molecular weight of the synthesized polymer may be determined by the reaction conditions (e.g., temperature, starting materials, concentration, solvent, etc.) used in the synthesis.

In another embodiment, one or more types of amine monomers and/or diacrylate monomers may be used in the polymerization reaction. For example, a combination of ethanolamine and ethylamine may be used to prepare a polymer more hydrophilic than one prepared using ethylamine alone, and also more hydrophobic than one prepared using ethanolamine alone.

A synthesized polymer may be purified by any technique known in the art including, but not limited to, precipitation, crystallization, chromatography, etc. In one embodiment, the polymer may be purified through repeated precipitations in organic solvent (e.g., diethyl ether, hexane, etc.). In another embodiment, the polymer may be isolated as a salt (e.g., a hydrochloride salt).

As described above, a variety of nanostructures can be used in association with the composites described herein. In some embodiments, carbon-based nanostructures are described. As used herein, a “carbon-based nanostructure” comprises a fused network of aromatic rings wherein the nanostructure comprises primarily carbon atoms. In some instances, the nanostructures have a cylindrical, pseudo-cylindrical, or horn shape. A carbon-based nanostructure can comprises a fused network of at least about 10, at least about 50, at least about 100, at least about 1000, at least about 10,000, or, in some cases, at least about 100,000 aromatic rings. Carbon-based nanostructures may be substantially planar or substantially non-planar, or may comprise a planar or non-planar portion. Carbon-based nanostructures may optionally comprise a border at which the fused network terminates. For example, a sheet of graphene comprises a planar carbon-containing molecule comprising a border at which the fused network terminates, while a carbon nanotube comprises a nonplanar carbon-based nanostructure with borders at either end. In some cases, the border may be substituted with hydrogen atoms. In some cases, the border may be substituted with groups comprising oxygen atoms (e.g., hydroxyl). In other cases, the border may be substituted as described herein.

In some embodiments, the nanostructures described herein may comprise nanotubes. As used herein, the term “nanotube” is given its ordinary meaning in the art and refers to a substantially cylindrical molecule or nanostructure comprising a fused network of primarily six-membered rings (e.g., six-membered aromatic rings). In some cases, nanotubes may resemble a sheet of graphite formed into a seamless cylindrical structure. It should be understood that the nanotube may also comprise rings or lattice structures other than six-membered rings. Typically, at least one end of the nanotube may be capped, i.e., with a curved or nonplanar aromatic group. Nanotubes may have a diameter of the order of nanometers and a length on the order of microns, tens of microns, hundreds of microns, or millimeters, resulting in an aspect ratio greater than about 100, about 1000, about 10,000, or greater. In some embodiments, a nanotube can have a diameter of less than about 1 micron, less than about 500 nm, less than about 250 nm, less than about 100 nm, less than about 75 nm, less than about 50 nm, less than about 25 nm, less than about 10 nm, or, in some cases, less than about 1 nm.

In some embodiments, a nanotube may comprise a carbon nanotube. The term “carbon nanotube” refers to nanotubes comprising primarily carbon atoms. Examples of carbon nanotubes include single-walled carbon nanotubes (SWNTs), double-walled carbon nanotubes (DWNTs), multi-walled carbon nanotubes (MWNTs) (e.g., concentric carbon nanotubes), inorganic derivatives thereof, and the like. In some embodiments, the carbon nanotube is a single-walled carbon nanotube. In some cases, the carbon nanotube is a multi-walled carbon nanotube (e.g., a double-walled carbon nanotube).

In some embodiments, the nanostructures comprise non-carbon nanotubes. Non-carbon nanotubes may be of any of the shapes and dimensions outlined above with respect to carbon nanotubes. The non-carbon nanotube material may be selected from polymer, ceramic, metal and other suitable materials. For example, the non-carbon nanotube may comprise a metal such as Co, Fe, Ni, Mo, Cu, Au, Ag, Pt, Pd, Al, Zn, or alloys of these metals, among others. In some instances, the non-carbon nanotube may be formed of a semi-conductor such as, for example, Si. In some cases, the non-carbon nanotubes may be Group II-VI nanotubes, wherein Group II consists of Zn, Cd, and Hg, and Group VI consists of O, S, Se, Te, and Po. In some embodiments, non-carbon nanotubes may comprise Group III-V nanotubes, wherein Group III consists of B, Al, Ga, In, and TI, and Group V consists of N, P, As, Sb, and Bi. As a specific example, the non-carbon nanotubes may comprise boron-nitride nanotubes.

In some embodiments, the nanotube may comprise both carbon and another material. For example, in some cases, a multi-walled nanotube may comprise at least one carbon-based wall (e.g., a conventional graphene sheet joined along a vector) and at least one non-carbon wall (e.g., a wall comprising a metal, silicon, boron nitride, etc.). In some embodiments, the carbon-based wall may surround at least one non-carbon wall. In some instances, a non-carbon wall may surround at least one carbon-based wall.

A composite may comprise use or addition of one or more binding materials or support materials. The binding or support materials may be polymeric materials, fibers, metals, or other materials. Polymeric materials for use as binding materials and/or support materials may be any material compatible with nanostructures.

As described herein, a composite may be configured to release an active agent. In some embodiments, a composite may be loaded with an active agent. The active agent may be selected from organic compounds, inorganic compounds, proteins, nucleic acids, and/or carbohydrates. In some cases, the active agent may be a pharmaceutical agent (e.g., a drug). Suitable drugs include, but are not limited to, growth factors; angiogenic agents; anti-inflammatory agents; anti-infective agents such as antibacterial agents, antiviral agents, antifungal agents, and agents that inhibit protozoan infections; antineoplastic agents; anesthetics; anti-cancer compositions; autonomic agents; steroids (e.g., corticosteroids); non-steroidal anti-inflammatory drugs (NSAIDs); antihistamines; mast-cell stabilizers; immunosuppressive agents; antimitotic agents; vaccines; diagnostic agents; or other drugs.

In some embodiments, a device may be loaded with an active agent by soaking the device in a solution containing the active agent. Generally, the loading of an active agent can be increased by increasing the concentration of the active agent in the soaking solution and/or increasing the contact time between the device and the soaking solution. An active agent may also adsorb onto the surface of the device. The association of an active agent with a device may result from non-covalent interactions. Alternatively, an active agent may be reacted with a functional group in the composite to form a covalent bond. As known to those in the art, a covalent bond may be chosen such that under certain conditions (e.g., physiological conditions), the bond may break thereby releasing the active agent. Depending on the ratio of the active agent to the polymer in the composite, the nature of the particular polymer employed, and the type of association between the active agent and the polymer, the rate of release of the active agent can be controlled.

In some embodiments, a virus and/or cell may be delivered using the composite. For example, the composite may be constructed to have a porous scaffold structure that can contain viruses and/or cells. The composite may be configured such that the virus and/or cell can be released in sustained fashion. In some cases, a virus may be used for gene delivery. Gene delivery may be beneficial, for example, for transforming non-proliferative cells into proliferative cells. A cell may be used, in some instances, as an active agent factory. For example, a cell (e.g., a stem cell) may secrete a growth factor or other agent that has therapeutic value. By placing such cells proximate a desired zone of treatment in a subject, these cells may continuously generate and deliver a therapeutic.

The active agents described herein may be used in “pharmaceutical compositions” or “pharmaceutically acceptable” compositions, which comprise a therapeutically effective amount of an active agent associated with one or more of the composites described herein, formulated together with one or more pharmaceutically acceptable carriers, additives, and/or diluents. The pharmaceutical compositions described herein may be useful for diagnosing, preventing, treating or managing a disease or bodily condition.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The amount of active agent which can be combined with a composite to produce a single dosage form will vary depending upon the host being treated, and the particular mode of administration. The amount of active agent that can be combined with a composite to produce a single dosage form will generally be that amount of the compound which produces a therapeutic effect. Generally, this amount will range from about 1% to about 99% of active ingredient, from about 5% to about 70%, or from about 10% to about 30%. It should be understood that ranges outside these ranges may be used as well.

Active agents described herein may be formulated as a solution, dispersion, or a suspension in an aqueous or non-aqueous liquid, as an emulsion or microemulsion (e.g., an oil-in-water or water-in-oil liquid emulsion), or as an elixir or syrup, or as pastilles (using an inert base, such as gelatin and glycerin, or sucrose and acacia), each containing a predetermined amount of the active agent.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A liquid dosage form may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Suspensions, in addition to an active agent, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The composites described herein may also contain excipients such as preservatives, wetting agents, emulsifying agents, lubricating agents and dispersing agents. Prevention of the action of microorganisms upon the composites may be facilitated by the inclusion of various antibacterial and antifungal agents, for example, paraben, chlorobutanol, phenol, sorbic acid, and the like. It may also be desirable to include isotonic agents, such as sugars, sodium chloride, and the like into the composites.

Delivery systems suitable for use with devices described herein include time-release, delayed release, sustained release, or controlled release delivery systems. Many types of release delivery systems are available and known to those of ordinary skill in the art. Specific examples include, but are not limited to, erosional systems in which the composition is contained in a form within a matrix, or diffusional systems in which an active component controls the release rate. The compositions may be as, for example, particles (e.g., microparticles, microspheres, nanoparticles), hydrogels, polymeric reservoirs, or combinations thereof. In some embodiments, the system may allow sustained or controlled release of an active agent to occur, for example, through control of the diffusion or erosion/degradation rate of the formulation or particle. The composites described herein can also be combined (e.g., contained) with delivery devices such as syringes, catheters, tubes, and implantable devices.

When the composites described herein are administered as pharmaceuticals, to humans and animals, they can be given per se or as a pharmaceutical composition containing, for example, about 0.1% to about 99.5%, about 0.5% to about 90%, or the like, of drug release material in combination with a pharmaceutically acceptable carrier.

An active agent may be given in dosages, e.g., at the maximum amount while avoiding or minimizing any potentially detrimental side effects. The active agents can be administered in effective amounts, alone or in a combinations with other compounds. For example, when treating cancer, a composition may include a cocktail of compounds that can be used to treat cancer.

The phrase “therapeutically effective amount” as used herein means that amount of a material or composition which is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

In some embodiments, the effective amount of any drug release described herein may be from about 1 ng/kg of body weight to about 10 mg/kg of body weight, and the frequency of administration may range from once a day to a once a month basis, to an as-needed basis. However, other dosage amounts and frequencies also may be used as the invention is not limited in this respect. A subject may be administered devices described herein in an amount effective to treat one or more diseases or bodily conditions described herein.

The effective amounts will depend on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose can be used, that is, the highest safe dose according to sound medical judgment.

The selected dosage level can also depend upon a variety of factors including the activity of the particular inventive structure employed, the route of administration, the time of administration, the rate of excretion or metabolism of the materials or active agents being employed, the duration of the treatment, other drugs, compounds and/or materials used in combination with the particular material employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the agents described herein employed in the pharmaceutical composition at levels lower than that required to achieve the desired therapeutic effect and then gradually increasing the dosage until the desired effect is achieved.

In some embodiments, a device or pharmaceutical composition described herein is provided to a subject chronically. Chronic treatments include any form of repeated administration for an extended period of time, such as repeated administrations for one or more months, between a month and a year, one or more years, or longer. In many embodiments, a chronic treatment involves administering a device or pharmaceutical composition repeatedly over the life of the subject. For example, chronic treatments may involve regular administrations, for example one or more times a week, or one or more times a month.

As used herein, a “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the devices are directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

While it is possible for an active agent to be administered alone, it may be administered as a pharmaceutical composition as described above.

In one embodiment, a kit may be provided, containing one or more of the above compositions. A “kit,” as used herein, typically defines a package or an assembly including one or more of the compositions of the invention, and/or other compositions associated with the invention, for example, as previously described. A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Any of the above-mentioned compositions useful for diagnosing, preventing, treating, or managing a disease or bodily condition may be packaged in kits, optionally including instructions for use of the composition. That is, the kit can include a description of use of the composition for participation in any disease or bodily condition. The kits can further include a description of use of the compositions as discussed herein. Instructions also may be provided for administering the composition by any suitable technique.

The kits described herein may also contain one or more containers, which can contain components such as the composites and/or active agents. The kits also may contain instructions for preparing and/or administrating the composites. The kits also can include other containers with one or more solvents, surfactants, preservatives, and/or diluents (e.g., normal saline (0.9% NaCl), or 5% dextrose) as well as containers for preparing and/or administering the composites to the patient in need of such treatment.

The compositions of the kit may be provided as any suitable form, for example, essentially dry or at least partially hydrated. When essentially dry, the composition may be hydrated by the addition of a suitable solution, which may also be provided. In embodiments where at least partially hydrated forms of the composition are used, the liquid form may be concentrated or ready to use.

The kit, in one set of embodiments, may comprise one or more containers such as vials, tubes, syringes, and the like, each of the containers comprising one or more of the elements to be used in the method. For example, one of the containers may contain a composite. Additionally, the kit may include containers for other components, for example, solutions to be mixed with the composite prior to administration.

U.S. Provisional Patent Application No. 61/286,764, filed Dec. 15, 2009, and entitled “Degradable Polymer Nanostructures;” U.S. Pat. No. 7,427,394, entitled “Biodegradable Poly(Beta-Amino Esters) and Uses Thereof,” issued Sep. 23, 2008; U.S. Pat. No. 6,998,115, entitled “Biodegradable Poly(β-amino esters) and Uses Thereof,” issued Feb. 14, 2006; U.S. patent application Ser. No. 11/758,078, filed Jun. 5, 2007, entitled “Crosslinked, Degradable Polymers and Uses Thereof,” published as U.S. Patent Application Publication No. 2008/0145338 on Jun. 19, 2008; U.S. patent application Ser. No. 11/780,754, filed Jul. 21, 2006, entitled “End-Modified Poly(beta-amino esters) and Uses Thereof,” published as U.S. Patent Application Publication No. 2008/0242626 on Oct. 2, 2008; U.S. patent application Ser. No. 11/099,886, filed Apr. 6, 2005, entitled “Biodegradable Poly(Beta-Amino Esters) and Uses Thereof,” published as U.S. Patent Application Publication No. 2005/0265961 on Dec. 1, 2005; U.S. patent application Ser. No. 12/568,481, Sep. 28, 2009, entitled “Biodegradable Poly(Beta-Amino Esters) and Uses Thereof”; and U.S. patent application Ser. No. 12/833,749, filed Jul. 9, 2010, entitled “Biodegradable Poly(Beta-Amino Esters) and Uses Thereof,” are incorporated herein by reference in their entirety for all purposes. All patents and patent applications mentioned herein are incorporated herein by reference in their entirety for all purposes.

The following examples are intended to illustrate certain embodiments of the present invention, but do not exemplify the full scope of the invention.

### EXAMPLES

### Example 1

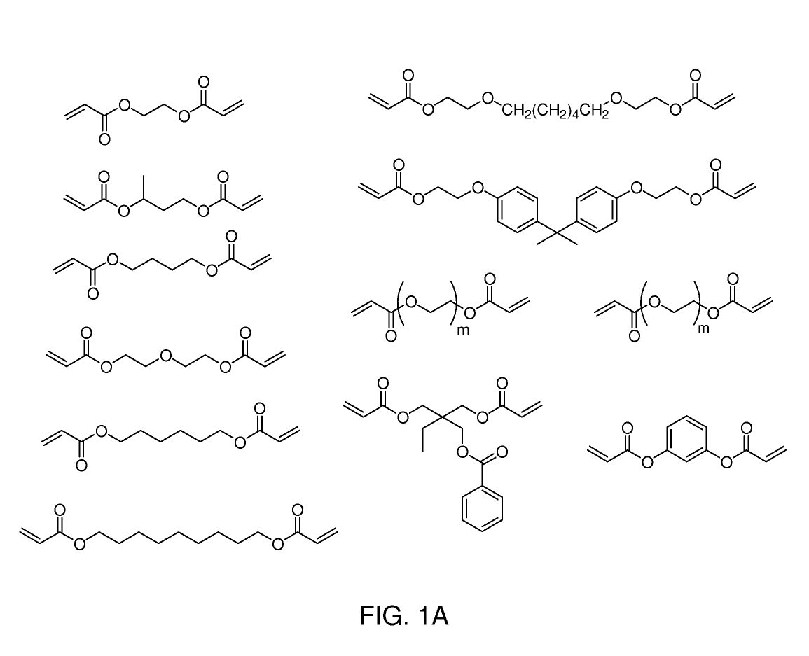

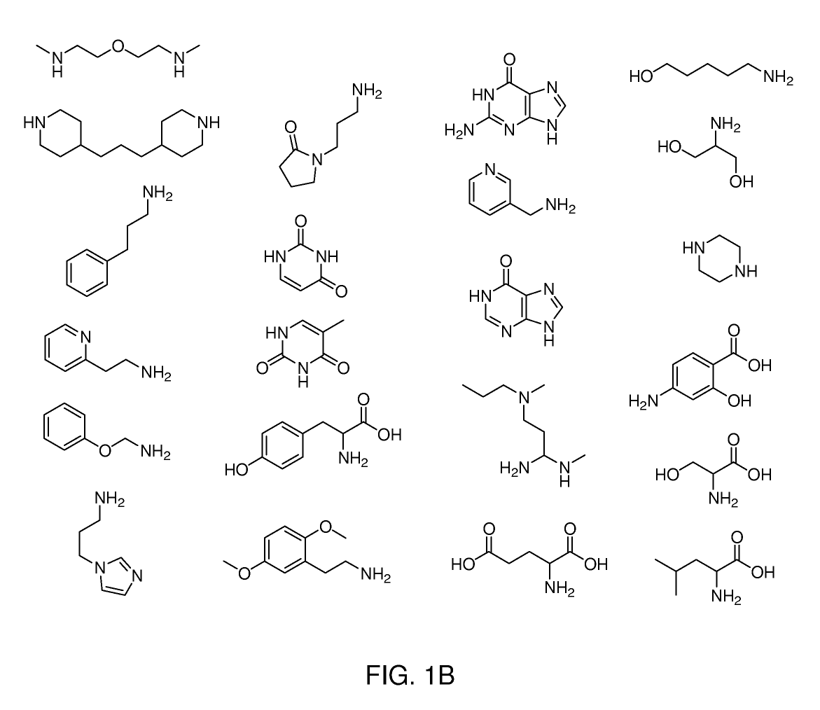

This example demonstrates synthesis of poly(beta-amino ester) polymers. Poly(beta-amino esters) (PBAE) were synthesized on a 250 microliter scale using a modified previously described polymerization method. Specifically, 0.5 mmole of each diacrylate was reacted with an essentially equivalent amount of either a bis-secondary diamine or a primary amine. All reactions were carried out in acetonitrile at 50° C. for 24 hours with the aid of the Symyx Core Module (Symyx Technologies, Inc., Sunnyvale, Calif.). For scale-up syntheses, the PBAE reactions were run at equivalent conditions but on a 20 mmole scale. A 264 PBAE polymer library was synthesized using 12 diacrylates (FIG. 1A) and 22 amines (FIG. 1B).

### Example 2

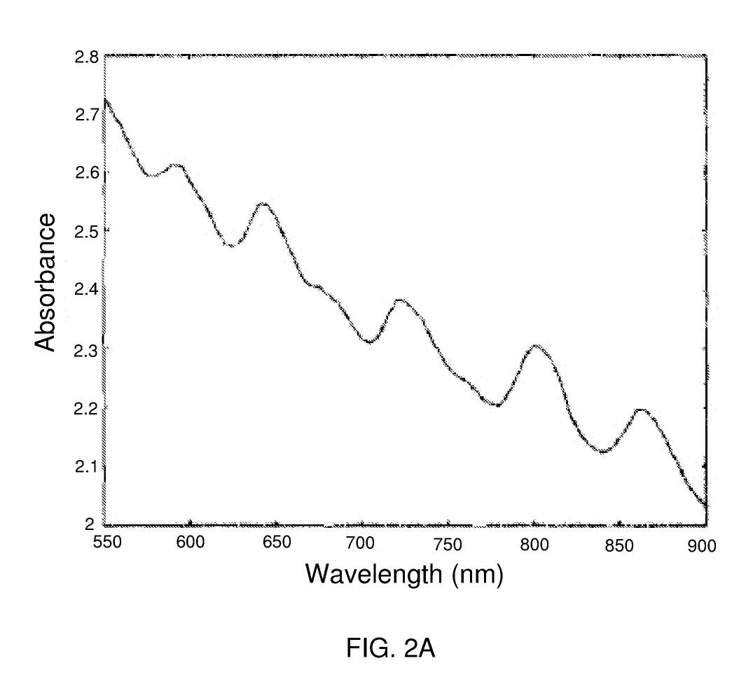

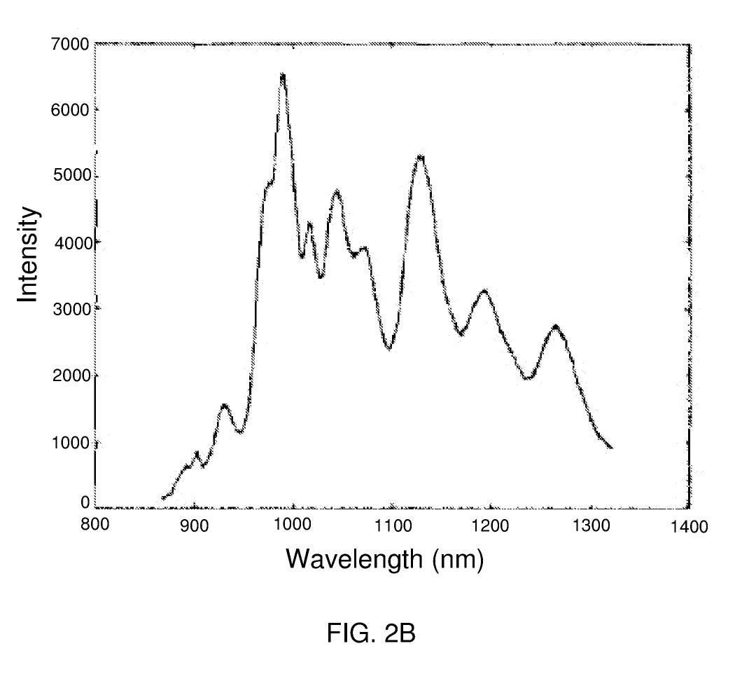

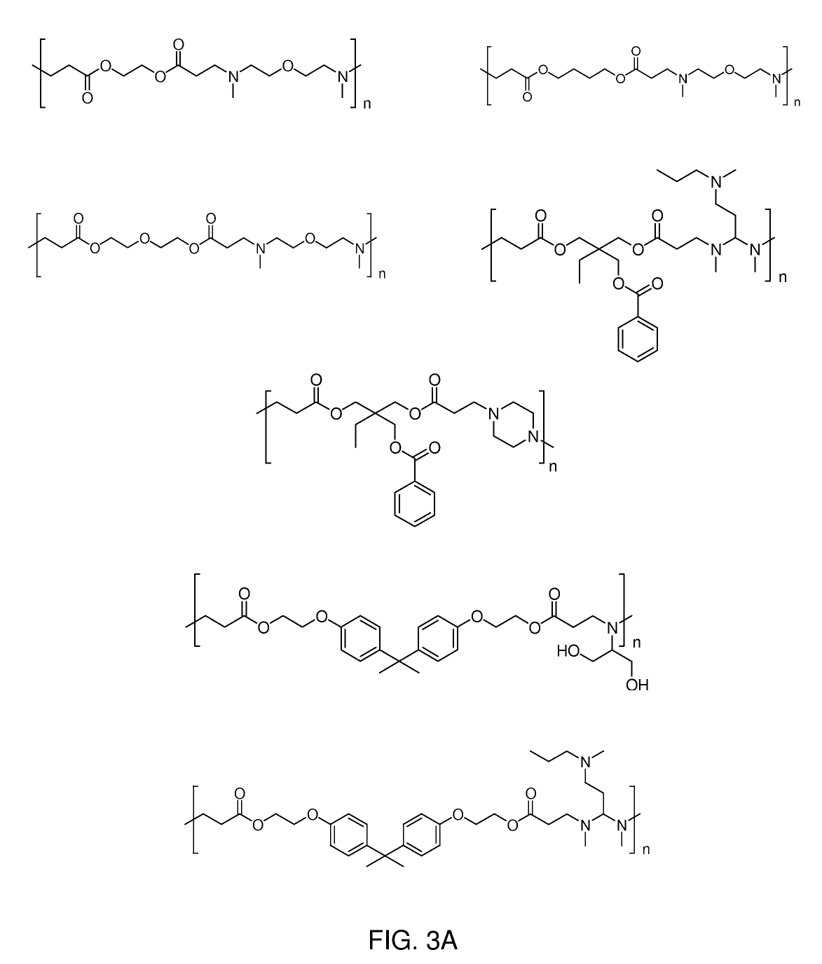

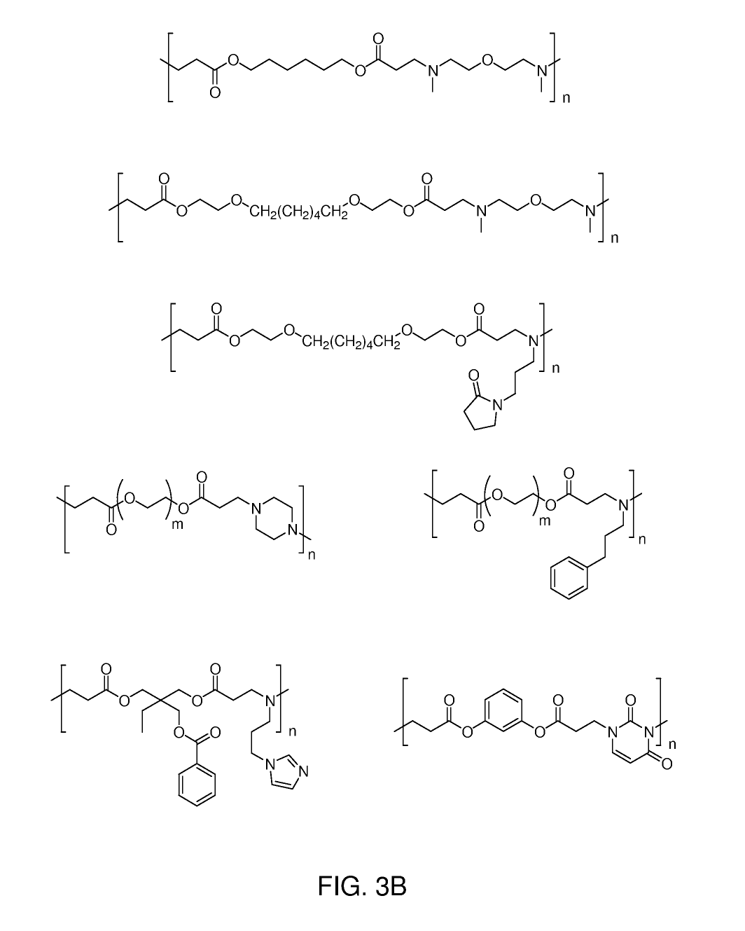

This example demonstrates preparation of nanostructure/polymer composites. In order to evaluate the ability of synthesized polymers to wrap individual single-walled carbon nanotubes (SWNT), all polymers were tested in various solvents (water, butanol, acetonitrile, chloroform, methylene chloride, DMF, etc.). Firstly, the synthesized polymers (˜10 mg/well) and SWNT (˜0.1 mg/well) were aliquoted into a glass 96-well plate giving a polymer:SWNT ratio of 100:1. All samples were probe-tip sonicated for 5 minutes at 25% amplitude and centrifuged at 4000 RPM for 10 minutes. The supernatant was removed and tested by UV-vis and fluorescence for the presence of individually wrapped SWNT. FIG. 2A and FIG. 2B shows representative data for a polymer-wrapped SWNT (or hit) as determined by UV-vis and fluorescence, respectively. Different polymer-wrapped SWNT in the various solvents were tested. For example, FIG. 3A and FIG. 3B show the structure of polymers that wrapped SWNT in the presence of water and acetonitrile, respectively. In this example, polymers that include aromatic chemical structures consistently wrapped SWNT. In addition, polymers having surfactant properties were also effective at wrapping SWNT. As used herein, a “polymer having surfactant properties” means a polymer that can reduce the interfacial tension between a fluid and a nanostructure.

### Example 3

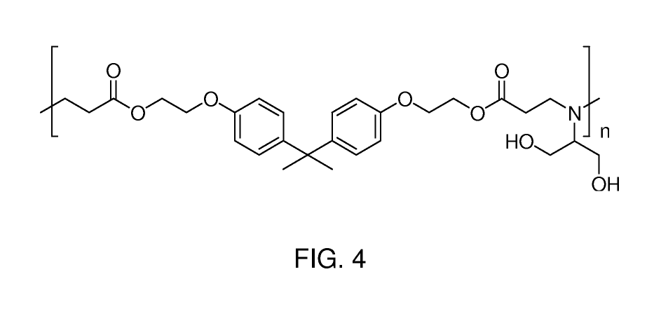

This example demonstrates fabrication of nanostructure/polymer composite films. Composite (polymer/SWNT) and polymer-only thin films were synthesized by layer-by-layer deposition (LbL). For example, bilayers of a PBAE polymer (cationic) and a poly(acrylic acid) (PA) polymer (anionic) were deposited onto either glass or Teflon substrates serially to produce a total of about 200 bilayers. After deposition, the films were dried with nitrogen gas. For films containing SWNT, every tetra layer included PBAE-wrapped SWNT rather than PBAE alone. Table 1 below provides film thickness and modulus data for polymer 153 (the structure of which is illustrated in FIG. 4) with and without SWNT as determined by profilometry and nanoindentation, respectively. The film modulus increased over 6-fold with less than 0.025 wt % SWNT loading in the composite.

Indentation experiments were conducted in ambient air using a pendulum-based instrumented nanoindenter. Samples were indented with a 5 micrometer tip at n=10 locations for each polymer film. The thickness correction method described in Constantinides, G., et. al., “Grid Indentation Analysis of Composite Microstructure and Mechanics: Principles and Validation,” Materials Science and Engineering A, 430, pp. 189-202 (2006) was used to adjust for the difference in thickness between the films with SWNTs and those without SWNTs.

Thickness measurements were made by scoring the films with a razor blade and measuring the step change in height between the film and substrate at n=5 locations for each polymer film with a Tencor P16 profilometer.

TABLE 1
LbL film thickness data.

| Film | Ave. thickness | | | |
|---|---|---|---|---|
| composition | (pm) | Ra (pm) | Ra (pm) | Er (GPa) |
| P153 | 0.806 | 0.203 | 0.319 | 3.02 ± 0.67 |
| P153-SWNT | 8.876 | 0.825 | 1.036 | 18.42 ± 4.85 |

### Example 4

This example demonstrates fabrication of nanostructure/polymer composite fibers. Nanostructure/polymer composite fibers were fabricated by essentially instantaneous complexation of cationic and anionic polymers in solution. For example, fibers comprising PBAE or PBAE/SWNT with PA can be formed from single phase (i.e., a one solution system containing both a cationic polymer and an anionic polymer) and two-phase systems (i.e., a two solution system, where a first solution contains a cationic polymer and a second solution contains an anionic polymer, and the first solution and the second solution are essentially immiscible with each other). In the case of a single phase system, the fibers were formed analogously to cotton candy where swirling a rod in the solution continuously grows the fibers. In the two-phase system, the fibers were formed at the interface between the first solution and the second solution, which is analogous to nylon fiber synthesis, where the fiber is continuously formed at the interface as the fiber is being removed. Both of these methods yielded fibers containing polymer-wrapped SWNT.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composite, comprising:
a polymer comprising a poly(beta-amino ester); and
a plurality of nanostructures contained within a volume of the polymer,
wherein the yield strength and/or effective Young's modulus of the composite is substantially greater than that which would be observed in the absence of the nanostructures, but under otherwise substantially identical conditions.

2. An article, comprising:
a fluid;
a polymer comprising a poly(beta-amino ester) contained within the fluid; and
a nanostructure contained within the fluid and interacting with the polymer.

3. A method, comprising:
providing a fluid;
distributing, within the fluid, a polymer comprising a poly(beta-amino ester);
distributing, within the fluid, a plurality of nanostructures.

4-6. (canceled)

7. The method of claim 3, wherein the polymer is solidified such that at least a portion of the nanostructures are contained within a volume of the polymer to form a composite.

8-9. (canceled)

10. The composite of claim 1, wherein the nanostructure is a nanotube, a nanofiber, and/or a nanowire.

11. (canceled)

12. The article of claim 2, wherein the fluid comprises a liquid.

13. (canceled)

14. The article of claim 2, wherein the fluid comprises an organic liquid.

15. (canceled)

16. The composite of claim 1, wherein the poly(beta-amino ester) is selected from the group consisting of: 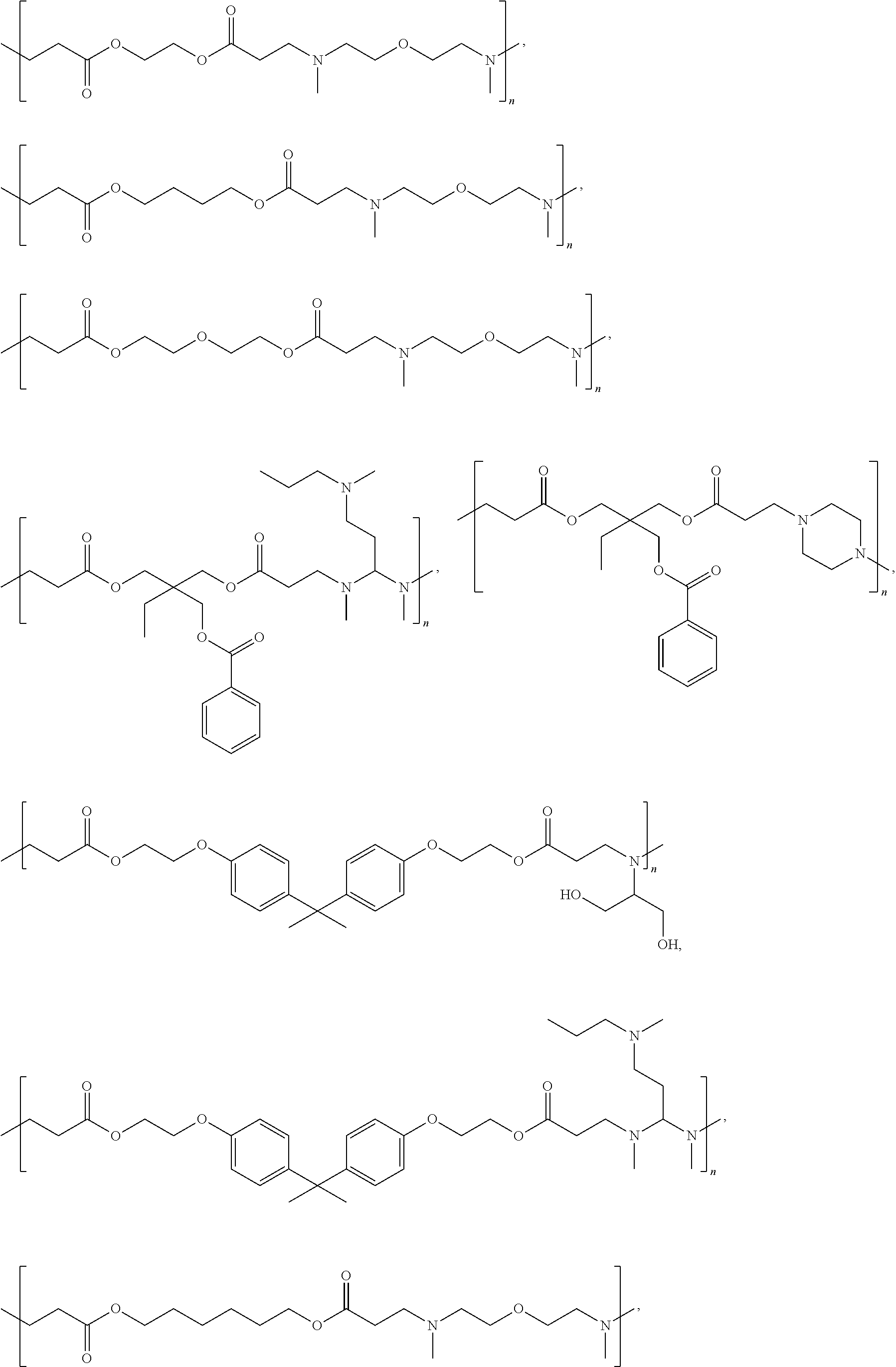 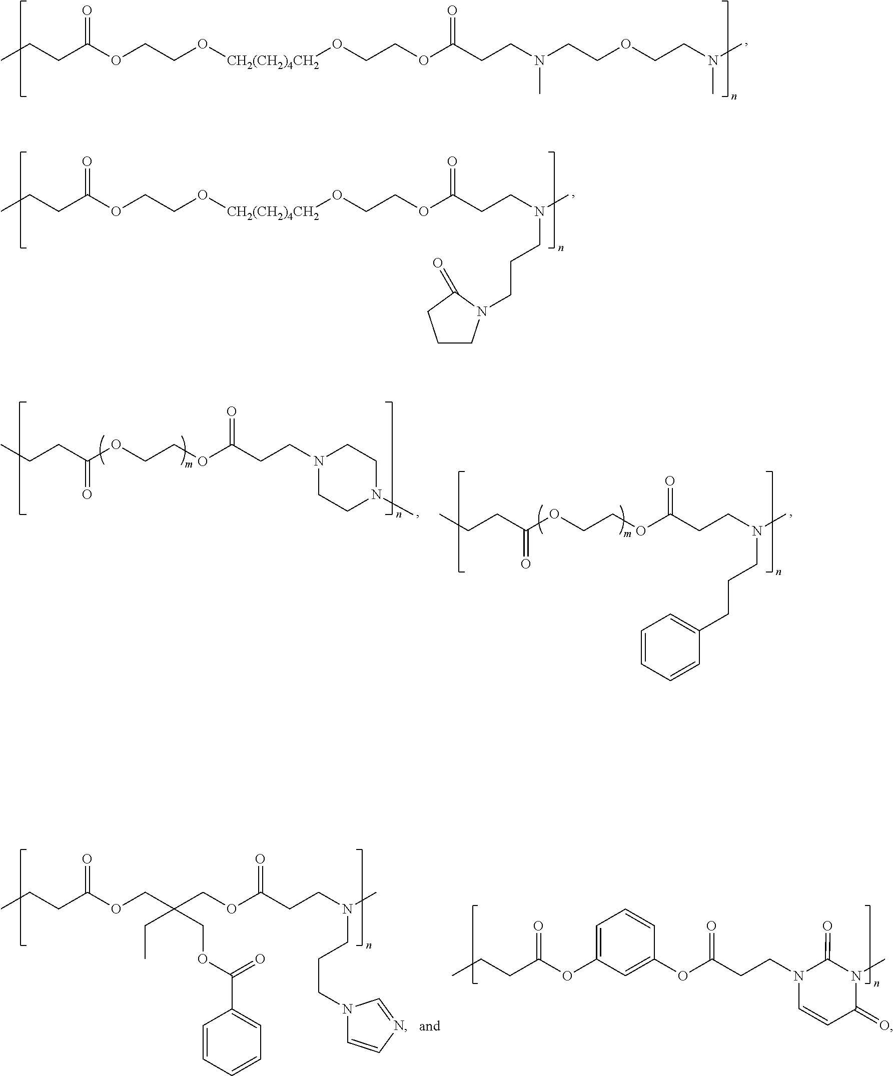
where n is at least 2 and m is at least 2.

17. The composite of claim 1, wherein the poly(beta-amino ester) comprises: 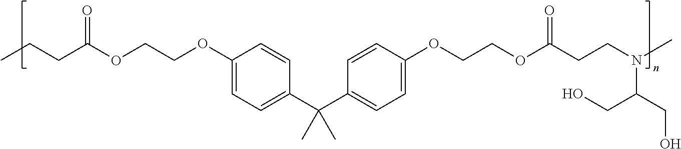
where n is at least 2.

18. (canceled)

19. The composite of claim 1, further comprising an anionic polymer.

20. (canceled)

21. The composite of claim 1, wherein the polymer comprises at least one aromatic ring.

22. The composite of claim 1, wherein the polymer comprises less than about 5 wt % aromatic rings.

23. The composite of claim 1, wherein the polymer is non-aromatic.

24-26. (canceled)

27. The composite of claim 1, wherein the composite is constructed using layer-by-layer deposition.

28. The composite of claim 1, wherein the composite comprises a plurality of fibers, the fibers comprising the polymer and the nanostructures, wherein the fibers are formed using a two-phase system.

29. The composite of claim 1, wherein the composite comprises a plurality of cells.

30. (canceled)

31. The composite of claim 1, wherein the effective Young's modulus of the composite is at least about 5 GPa.

32-33. (canceled)

34. The composite of claim 1, wherein the yield strength of the composite is at least about 100 MPa.

35-38. (canceled)

39. The composite of claim 1, wherein the composite is part of a medical device.

40. The composite of claim 39, wherein the medical device comprises an active agent.

41-43. (canceled)
